(19) United States
(12) Patent Application Publication
(10) Pub. No.: US 2026/0364386 A1
(43) Pub. Date: Nov. 19, 2026

(54) THERAPEUTIC ULTRASOUND METHODS

(71) Applicants: B.G. NEGEV TECHNOLOGIES AND APPLICATIONS LTD., Beer-Sheva (IL); THE JOHNS HOPKINS UNIVERSITY, Baltimore, MD (US)

(72) Inventors: Joseph KOST, Beer-Sheva (IL); Tamar TRAITEL, Beer-Sheva (IL); Riki GOLDBART, Beer-Sheva (IL); Nitsa BUARON, Beer-Sheva (IL); Antonella MANGRAVITI, Roma (IT); Henry BREM, Ellicott City, MD (US); Betty TYLER, Catonsville, MD (US)

(21) Appl. No.: 19/488,180

(22) PCT Filed: May 31, 2024
(86) PCT No.: PCT/IL2024/050543

§ 371 Date: Nov. 26, 2025

Related U.S. Application Data
(60) Provisional application No. 63/505,475, filed on Jun. 1, 2023.

(51) Int. Cl. A61N 7/02; A61N 7/00

(52) U.S. Cl. CPC A61N 7/022; A61N 2007/0026

(57) ABSTRACT

Provided herein methods of treating diseases or disorders, particularly primary brain cancers, brain metastases, and other cancers, as well as inflammatory disorders, by directly insonating tissues with low-frequency ultrasound.

### FIELD OF THE INVENTION

The present disclosure generally relates to methods of treatment of primary brain cancers, metastatic brain cancers, other cancers, and inflammatory conditions, based on the direct irradiation of a respective tissue or an organ with low-frequency ultrasound.

### BACKGROUND OF THE INVENTION

Ultrasound (US) is a sound wave with a frequency above the human hearing range (ca.>18 kHz). In biophysical settings, it has been shown to affect mass transport, enhance the release of encapsulated drugs, and to facilitate gene therapy (S. Mitragotri, Nat. Rev. Drug Discov. 4 (2005) 255-260. https://doi.org/10.1038/nrd1662). In addition to using US as a tool for improving the performance of therapeutic entities, it may be used by itself for various treatment applications, inter alia, certain instances of wound healing and treatment of inflammatory conditions, as well as treatment of some proliferative conditions (J. Hart, J. Wound Care. 7 (1998) 25-28. https://doi.org/10.12968/jowc.1998.7.1.25; J. I. Chung, et al., Osteoarthr. Cartil. 20 (2012) 314-322. https://doi.org/10.1016/j.joca.2012.01.005; P. Wang, et al., Exp. Ther. Med. 2 (2011) 849-852. https://doi.org/10.3892/etm.2011.317; and Q. Ye, et al., Ultrasound Med. Biol. 42 (2016) 2253-2260. https://doi.org/10.1016/j.ultrasmedbio.2016.04.017).

Interestingly, the biological effects of US observed on normal cells are different from those observed on cancer cells, as shown by several studies. For example, cancer cells were shown to be more susceptible to US than normal cells (F. Lejbkowicz, et al., Environ. Health Perspect. 1997, pp. 1575-1578. https://doi.org/10.2307/3433673; F. Lejbkowicz, et al., Ultrasound Med. Biol. 19 (1993) 75-82. https://doi.org/10.1016/0301-5629 (93) 90020-O; A. Azagury, et al., Ultrasound Med. Biol. 42 (2016) 1560-1567. https://doi.org/10.1016/j.ultrasmedbio.2016.02.005). However, this phenomenon is not entirely clear or consistent.

Additionally, Lagneaux et al. evaluated the cytotoxic effect of “low energy” US (0.007 W/mL acoustical power transmitted to cell) on normal and leukemic cells in vitro in terms of cell viability and apoptotic characteristics and reported that US induced apoptosis, specifically in leukemic cells, and offered oxidative stress as the mechanism inducing apoptosis (L. Lagneaux, et al., Exp. Hematol. 30 (2002) 1293-1301).

Non-invasive ultrasound insonation of mice bearing breast adenocarcinoma was reported in U.S. Pat. No. 10,960,233 and in the continuation application thereof, US patent application publication number 2021/0213307. Low intensity US was also suggested for the treatment of glioblastoma in combination with microbubbles (Z. Zhang, et al., Brain Res. 1473 (2012) 25-34. https://doi.org/10.1016/j.brainres.2012.06.047), wherein apoptosis was increased in tumor cells, and it was shown that US was able to trigger apoptotic signaling pathways by up- or down-regulating apoptotic related proteins levels. However, this study was aimed at improving the response to chemotherapy by increasing apoptosis and not at utilizing US per se, namely, as a standalone treatment.

Brain tumors represent one of the most malignant forms of human cancers, with dismal patient outcomes. A glioma is a primary brain tumor originating from glial-type cells in the brain or spinal cord and is often malignant. The most common and aggressive tumor is the grade IV astrocytoma, also known as glioblastoma multiforme (GBM) making up 57.3% of all gliomas and 48.3% of primary malignant brain tumors; the prognosis is often quite poor, and the average survival period with treatment is around 14 months. Despite recent progress in both surgical and imaging techniques, as well as in adjuvant therapies, the current treatment that includes surgical removal of accessible tumor followed by radio- and chemo-therapy offers poor prognosis with few long-term options. One of the main reasons for the low response rate is the difficulty to achieve complete surgical resection due to the infiltrating nature of GBM cells into the normal brain tissue as in the case of metastatic brain tumors. In addition, treating the non-resected margin of the tumor with current adjuvant therapies is not effective, due to low permeability of the drug from the application site, and further elicits concerns that these methods are non-selective and can cause systemic toxicity and damage to normal brain tissue. Metastatic brain tumors are typically treated with chemotherapy and more therapeutic options are necessary.

U.S. Pat. No. 7,717,853 describes a method for delivering ultrasound energy to a patient's intracranial space for use in the diagnostic or therapeutic treatment of acute or chronic neurological conditions.

There remains a need in the art for local therapy strategies that can treat the tumor and its diffusive margin while having minimal toxicity on healthy brain cells, and have the potential of significantly improving patient prognosis.

### SUMMARY OF THE INVENTION

The present disclosure provides an ultrasound-based therapeutic tool for treating several diseases and/or conditions, such as brain tumors and traumatic brain injury, using low frequency US protocols adjustable according to tumor nature and volume, optionally, without additional therapeutics.

Low frequency US may also be used as an adjuvant therapy, after surgical resection. US irradiation, which is directly applied (e.g., intra-cavity), may eliminate cancer cells from the healthy brain tissue, in a selective and effective manner, thus treating potentially also the non-resected margins of a tumor and diffusive or metastatic tumor cells. Without being bound by a particular theory, it is believed that ultrasound, in particular low frequency US, may penetrate deep into the tissue, deeper than locally applied cytotoxic agents, and therefore can be used advantageously for treating deep-seated invasive malignant tumors.

The present inventors have unexpectedly shown a high selectivity accompanied with high efficacy when US was used as a primary treatment tool (i.e., as a monotherapy) for treating a brain tumor in vivo, e.g., malignant glioma. Low frequency US, applied under conditions shown to be safe for healthy tissue, induced rapid inhibition of tumor growth. In addition, the present inventors have demonstrated that insonation specifically damaged the tumoral tissue, resulting in its necrosis, which was confined by a glial reaction. Remarkably, when US was applied more than once, the glial reaction was more progressed, and the gliosis was more extensive, such that the destruction of the tumor tissue was confirmed histologically. As shown below, one of the most important observations made was the minimal neurotoxicity caused by the US treatment: while the tumor tissue was destroyed by the US treatment, the healthy surrounding tissue was left unharmed, demonstrating that the tumor was eliminated in a selective manner. These results may pave the way for a therapeutic approach for treating malignant tumors (e.g., glioma) based on adjusting low frequency directly-applied US protocols according to the tumor volume, without requiring additional therapeutics.

It has been further unexpectedly found that inflammatory modulation of the brain tissue following an injury is possible. As demonstrated in the appended examples, following US treatment the leucocytic level at the lesion was reduced while the gliosis level increased, indicating an acceleration of the healing process and prevention of the spreading of the injured area. These results indicate that it may be possible to use direct US insonation in treatment of brain lesions, e.g., following a trauma or a stroke, particularly as adjuvant therapy to surgical procedures routinely performed for these conditions.

Therefore, by a first aspect thereof the present disclosure provides a method of treating a subject suffering from a primary cancer of a brain, said method comprising directly irradiating the cancerous lesion or non-resected tumor margins in a post-operative cavity with ultrasound having a frequency of between 20 and 200 kHz, at an ultrasound intensity of between 1 and 50 W/cm², and wherein said ultrasound intensity is characterized by a thermal index (TI) value of between 0.1 than 5 and a low-frequency mechanical index (LFMI) value between 0.15 than 3. The utilization of ultrasound with the mechanical index exceeding 3, e.g., up to 5, is also envisaged. Preferably, the direct irradiating is performed by placing the ultrasound transducer onto the cancerous lesion or into the post-operative cavity. Preferably, the method may be performed, wherein the frequency may be between about 20 kHz and 35 kHz. Further preferably, the intensity may be between about 1 and 5.5 W/cm² and the frequency may be between about 20 and 22 kHz. The ultrasound may be applied at least once for a time interval of between 5 seconds and 30 minutes. Optionally, the frequency and intensity are selected such that the thermal index value is selected from the group consisting of between 0.1 and 5.0, between 0.1 and 4.5, between 0.1 and 4.0, between 0.1 and 3.5, between 0.1 and 3.0, between 0.1 and 2.5, between 0.1 and 2.0, between 0.1 and 1.5, and between 0.1 and 1.0, and said low-frequency mechanical index value is selected from the group consisting of between 0.15 and 3.0, between 0.15 and 2.8, between 0.15 and 2.6, between 0.15 and 2.5, between 0.15 and 2.4, between 0.15 and 2.2, between 0.15 and 2.0, and between 0.15 and 1.9, although the low-frequency mechanical index of between 0.15 and 5, or between 0.15 and 4.5, or between 0.15 and 4, or between 0.15 and 3.5, are also envisaged under some conditions. The primary brain cancer may be selected from diseases or disorders classified in a subclass 02A00, 02A01, 02A02, or 02A0Z, of the 11th revision of World Health Organization International Classification of Diseases.

In a further aspect thereof the present disclosure provides a method of treating a subject suffering from a brain injury, the method comprising directly irradiating an area of the brain injury with ultrasound having a frequency of between 20 and 200 kHz, at an ultrasound intensity of between 0.3 and 50 W/cm², wherein the ultrasound intensity may be characterized by a thermal index (TI) value lower than 5.0 and a low-frequency mechanical index (MI) value lower than 3, although ultrasound having mechanical index of up to 5 is also envisaged. Optionally, the thermal index value may be selected from the group consisting of lower than 5.0, lower than 4.5, lower than 4.0, lower than 3.5, lower than 3.0, lower than 2.5, lower than 2.0, lower than 1.5 and lower than 1.0, and the low-frequency mechanical index value is selected from the group consisting of lower than 5.0, e.g., lower than 4.5′ or lower than 4.0, or lower than 3.5, or lower than 3.0, lower than 2.8, lower than 2.6, lower than 2.5, lower than 2.4, lower than 2.2, lower than 2.0, and lower than 1.9. Optionally, the brain injury results from traumatic brain injury.

In a further aspect thereof the present disclosure provides a method of treating a subject suffering from a cancer in an organ or a tissue, said method comprising directly irradiating said organ or tissue with ultrasound having a frequency of between 20 and 200 kHz, at an ultrasound intensity of between 1 and 50 W/cm² and wherein said ultrasound intensity is characterized by a thermal index (TI) value of higher than 0.1 and lower than 5 and a low-frequency mechanical index (MI) value of higher than 0.15 and lower than 3. In some embodiments the direct irradiation may be performed by placing the ultrasound transducer in the proximity of said tissue or organ. In other embodiments the direct irradiation is performed by placing the ultrasound transducer within a cavity formed by or during a surgery in the vicinity of said organ or tissue.

In particular embodiments the method of the present disclosure is applied using frequency of between 20 kHz and 35 kHz. In further embodiments the method of the present disclosure is applied using intensity of between about 1 and 5.5 W/cm² and frequency of about 20 kHz, e.g., between 18 and 22 kHz, or between 20 and 22 kHz. In still further embodiments the method of the present disclosure is applied using intensity of between 4 and 5 W/cm² and frequency of about 20 kHz, e.g., between 18 and 22 kHz, or between 20 and 22 kHz.

In some embodiments of the method as herein defined, ultrasound is applied at least once for a time interval of between 5 seconds and 30 minutes. Within the methods of the present disclosure, ultrasound may be applied multiple times, at a regimen frequency of between once or twice daily to once weekly, during a period of between one to four weeks. In particular embodiments of the method of treatment as herein defined, ultrasound regimen of once or twice daily or once weekly during a period of between one to four weeks may be repeated, e.g. after a treatment intermission of about a month, two months, or more.

In the above and other embodiments of the method as herein defined, ultrasound is applied essentially at a perpendicular axis to said organ or tissue, e.g., at an angle of between about −60° and about 60°, preferably at an angle of between about −30° and about 30° relative to a perpendicular axis to said organ or tissue surface.

In some embodiments, the method according to the present disclosure is suitable as an adjuvant therapy to surgery of the organ or tissue as herein defined. In other embodiments, the method according to the preset disclosure is suitable for eliminating non-resected margins of cancer in the organ or tissue as herein defined after said surgery or for eliminating metastatic cells. By way of example, the cancer may be a metastasis of a cancer of different origin. By a further example, the method as herein defined is suitable for treatment of cancer selected from disease or disorders classified in any one of subclasses 02A20 to 02F9Z, of the 11th revision of World Health Organization International Classification of Diseases, preferably selected from the group consisting of sarcoma, carcinoma, lymphoma, and cancer of the breast, lung, prostate, colon, skin, and bladder. Optionally, the thermal index value may be selected from the group consisting of between 0.1 and 5.0, between 0.1 and 4.5, between 0.1 and 4.0, between 0.1 and 3.5, between 0.1 and 3.0, between 0.1 and 2.5, between 0.1 and 2.0, between 0.1 and 1.5 and between 0.1 and 1.0, and the low-frequency mechanical index value is selected from the group consisting of between 0.15 and 5.0, between 0.15 and 4.5, between 0.15 and 4.0, between 0.15 and 3.5, 0.15 and 3.0, between 0.15 and 2.8, between 0.15 and 2.6, between 0.15 and 2.5, between 0.15 and 2.4, between 0.15 and 2.2, between 0.15 and 2.0, and between 0.15 and 1.9.

### BRIEF DESCRIPTION OF THE DRAWINGS

Embodiments will now be described, by way of non-limiting example only, with reference to the accompanying drawings, in which:

FIG. 1A-FIG. 1B demonstrate a schematic drawing of the in vitro US experimental set-up, and, the bar graph showing C6 cell viability after exposure to different US conditions compared to control cells (under no US exposure), respectively.

FIG. 2A-FIG. 2C: a schematic workflow of the safety experiment, a quantification of spontaneous pain at different time points post-US irradiation at different conditions, and a graph showing weight change at different time points post US irradiation at different conditions, respectively.

FIG. 3A-FIG. 3F: FIG. 3A is a schematic workflow of the experiment studying the US effect on intracranial 9 L gliosarcoma tumor in vivo. FIG. 3B is an illustration of the in vivo experimental setup of US (20 kHz) application FIG. 3C is a bar graph showing the effect of US (at 4.6 W/cm² for 5 minutes) on 9 L gliosarcoma tumor growth. FIG. 3D shows a micrograph of coronal section of intracranially implanted 9 L gliosarcoma tumor in a F344 rat. FIG. 3E is a graph showing the dependence of US application on tumor volume FIG. 3F is a graph showing the dependence of US application effect on the tumor and US probe dimensions.

FIG. 4A-FIG. 4B demonstrate histopathological evaluations of tumor elimination at different time point, by different US protocols, with ultrasound being applied once or twice, respectively.

FIG. 5A-FIG. 5B demonstrate additional characteristics of the histopathological evaluations by the different US protocols.

FIG. 6A-FIG. 6D show representative H&E staining images of cortical lesion sites of rodent brain, seven days after injury from skull drilling, with no US or with US application (at 4.2 W/cm² for 5 minutes) 10-60 minutes after injury.

FIG. 7A-FIG. 7B: show representative H&E staining images of the gliosis region of cortical lesion site of rodent brain seven days after injury from skull drilling, with no US or with US application (at 4.2 W/cm² for 5 minutes) 10-60 minutes after injury.

FIG. 8A-FIG. 8B: show representative immunohistochemical staining images of the neovascularization (CD34 positive area) at the cortical lesion site of rodent brain seven days after injury from skull drilling, with no US or with US application (at 4.2 W/cm² for 5 minutes) 10-60 minutes after injury.

FIG. 9A-FIG. 9B: show representative H&E staining images of the granulation tissue formed at the cortical lesion site of rodent brain seven days after injury from skull drilling, with no US or with US application (at 4.2 W/cm² for 5 minutes) 10-60 minutes after injury.

### DETAILED DESCRIPTION OF THE INVENTION

Experimental studies conducted in the framework of the present disclosure have shown that ultrasound may be applied as an efficient and selective therapeutic tool for treating brain tumors, specifically malignant glioma, with minimal side effects. As demonstrated in the Examples below, low frequency ultrasound inhibited glioma cells proliferation in vitro. In addition, low frequency ultrasound applied under conditions found to be safe for healthy brain tissue, rapidly inhibited malignant glioma tumor growth in vivo in tumor-bearing rats, with complete destruction of tumor tissue while leaving the healthy surrounding tissue essentially unharmed.

These results were accompanied by an extensive anti-tumor immune response, which, without being bound by any theory, was based on glial reaction, also involving infiltration of glia cells from the healthy neural tissue surrounding the tumor into the tumor area, and enriching the tumour's microenvironment with pro-inflammatory cues which in turn stimulated an additional anti-tumoral immune response. The rapid immune response observed after US application allows the prevention of side effects associated with necrosis as the affected necrotic area is completely eliminated from the brain parenchyma, indicating a potentially promising anti-tumor treatment.

Remarkably, US was also found to have a beneficial effect on brain tissue injury, in vivo. The experimental results presented in the Examples below show decrease in the lesion area and the necrotic area in US-treated groups as compared to the control group. Morphometry analysis of brain sections showed a decrease in the number of lymphocytes and an increase of gliosis and increase in vascular density in US-treated groups compared to the control group. In addition, the amount of granulation tissue was found to be higher in US-treated groups compared to the control group. These results indicate that US may also be a promising tool for the treatment of brain injury.

Therefore, in a first aspect thereof the present disclosure provides a method of treating a subject suffering from a disease or a condition in an organ or a tissue, said method comprising directly irradiating said organ or tissue with ultrasound having a frequency of between 20 and 200 kHz, at an ultrasound intensity of between 0.3 and 50 W/cm². Preferably, the method involves direct irradiation of the tissue with ultrasound. Particularly preferably, the tissue is a brain tissue. Alternatively or additionally, the tissue may also be the adjacent tissue to the resection margin of a tumor. In some preferred embodiments, the intensity is between 1 and 50 W/cm², provided that the frequency and the intensity are selected to keep the insonation within the predefined ranges of thermal and low-frequency mechanical indices, as elaborated below.

In particular embodiments, and as detailed and exemplified herein, the present disclosure relates to treatment of cancer. In other words, the present disclosure provides a method of treating a subject suffering from a cancer in an organ or a tissue, said method comprising directly irradiating said organ or tissue with ultrasound having a frequency of between 20 and 200 kHz, at an ultrasound intensity of between 0.3 and 50 W/cm².

As used herein the term “irradiating” refers to ultrasonic stimulation or insonation of a cell, a tissue or an organ. In the field of diagnostic ultrasound, ultrasound at intensities lower than a threshold intensity (also referred to as “cavitational threshold intensity”) are preferred in order to minimize tissue interaction. However, therapeutic ultrasound, as in this case, is based on tissue interactions and may therefore sometimes benefit from an extension of this threshold. Although this extension has beneficial therapeutic effect, it may cause tissue damage not only to the cancerous tissue, but also to all cells of the neighboring surrounding healthy tissue that is also exposed to the irradiation to some extent. The threshold intensity depends on various parameters, such as organ/tissue type, coupling medium properties, frequency, organ/tissue temperature and gas content, to name but a few. However, as described herein, although the ultrasound intensity may be adjusted in such a way that it preferably remains below the threshold intensity, the irradiating step may preferably be performed such that the insonation remains within the limits of the thermal index and the low-frequency mechanical index as elaborated below.

Furthermore, the ultrasound treatment regimen, also referred to as an “ultrasound dose” or just “dose”, e.g., the delivered ultrasound intensity, includes a series of ultrasound parameters, including, but not limited to, ultrasound energy dose, ultrasound frequency, duty cycle, and treatment time. These specific ultrasound working parameters (collectively referred to as ultrasound dose) vary and may depend on a variety of factors, including the age, body weight, general health and sex of the individual being treated; other therapies and drugs that have been administered previously or in combination with the ultrasound therapy; and the type and severity of the particular condition (e.g. malignant disease) undergoing therapy.

Additional ultrasound working parameters include, but are not limited to, distance of transducer, area of transducer and additional parameters as detailed below, and as are known to a person skilled in the art.

Various safety measures are associated with the application of therapeutic US. As known in the art, the thermal index (TI) is a measure of the US thermal bio-effect, used in order to estimate the expected temperature rise due to acoustic energy exposure for assuring no thermal damage is caused. For soft tissues, the TI (TIs) is defined as the ratio of the emitted acoustic power to the power required to raise the tissue's temperature by 1 °C. According to the FDA safety guidelines, TI should be lower than unity (the value of 1, FDA, Information for Manufacturers Seeking Marketing Clearance of Diagnostic Ultrasound Systems and Transducers, Ultrasound. (2008) 1-64). The TIs may be calculated by equation 1 below:

$${TI}_{s} = \frac{{Wf}_{c}}{210} < 1 \qquad ( 1 )$$

where, W is the output power in mW, and f_{c} is the center frequency in MHz (W. D. O'Brien and D. Scott Ellis, IEEE Trans. Ultrason. Ferroelectr. Freq. Control. 46 (1999) 1459-1476. https://doi.org/10.1109/58.808870).

The mechanical index (MI) is a measure of the acoustic output and is an indicator for the probability of US mechanical (i.e. non-thermal) bioeffects. It is defined as the peak rarefactional pressure of an US longitudinal wave propagating in a uniform medium, divided by the square root of the center frequency of the transmitted US wave (W. D. O'Brien, Prog. Biophys. Mol. 93 (2007) Biol. 212-255. https://doi.org/10.1016/j.pbiomolbio.2006.07.010; J. B. Fowlkes and C. K. Holland, American Institute of Ultrasound in Medicine, in: J. Ultrasound Med., 2000: pp. 69-72. https://doi.org/10.7863/jum.2000.19.2.69). According to the FDA safety guidelines, MI should not exceed the value of 1.9 for diagnostic applications. The MI was calculated by equation 2:

$$MI = \frac{{P}_{r \text{.3}}}{\sqrt{{f}_{c}}} = \leq 1.9 \qquad ( 2 )$$

where, P_{r,3} is the derated peak rarefactional pressure in MPa, f_{c} is the center frequency in MHz, and P_{r,3} can be calculated using the US intensity and the acoustic impedance of soft tissue, e.g., as \sqrt{2*I*Z}×10⁻⁶, where I is the intensity in W/m², and Z is the acoustic impedance of soft tissue in kg/m² sec. To calculate the maximum MI in experiments involving rodents, Z of soft tissue ~1.6×10⁶ [Kg/m² sec] may be used (J. B. Fowlkes and C. K. Holland 2000, vide supra).

For low-frequency US, the adapted low-frequency mechanical index LF-MI can be calculated using an adjusted index (F. Ahmadi et al., Prog. Biophys. Mol. Biol., 108 (2012) 119-138, https://doi.org/10.1016/j.pbiomolbio.2012.01.004)) by equation 3:

$$MI = \frac{P - {P}_{0}}{\sqrt{{f}_{c}}} \leq 1.9 \qquad ( 3 )$$

where, P is the pressure amplitude in MPa, P₀ is the ambient pressure in MPa, f_{c} is the center frequency in MHz. Unless the context herein clearly dictates otherwise, the reference made to “mechanical index” should be construed as “low-frequency mechanical index”.

However, for the purpose of the present disclosure, it has been unexpectedly found that the thermal index of the US treatment may be kept between 0.1 and 5.0, and the low-frequency mechanical index may be kept below 5, e.g., between 0.15 and 3.0 according to the experimental upper limit of intensity and acoustic impedance (Z) two times higher than soft tissue.

Therefore, US intensities suitable for the present disclosure are characterized by a thermal index (TI) value of above 0.1, and lower than 5.0, preferably lower than 4.5, 4.0, 3.5, 3.0, 2.5, 2.0, 1.5 or lower than 1.0. Likewise, the low-frequency mechanical index (MI) value may be above 0.15, but lower than 5.0, e.g., lower than 4.5, or 4.0, or 3.5, or 3.0, e.g., lower than 2.8, 2.6, 2.5, 2.4, 2.2, 2.0 or lower than 1.9. In other words, the methods of the present disclosure are performed by applying ultrasound having a frequency of between 20 and 200 kHz, at an ultrasound intensity of between 0.3 and 50 W/cm², preferably between 1 and 50 W/cm², and such that the ultrasound intensity is characterized by a thermal index (TI) value lower than 5.0 and a low-frequency mechanical index (LF-MI) value lower than 5.0, preferably lower than 3.0.

The term “ultrasound intensity” is sometimes used interchangeably with the terms “intensity”, “power output”, “power density”, “power surface density”, “acoustic output”, and “spatial average intensity”, measured in Watts per unit area, usually in Watt/cm² (W/cm²) units. For example, for a frequency of 20 kHz, it was shown below that the ultrasound intensities may be up to 6.2 W/cm² (e.g., between about 0.3 and 5.5 W/cm² or up to 5 W/cm²). It is known that the cavitation intensity threshold is dependent on the US frequency, and at low-frequency ultrasound the cavitational threshold is the lowest. It has been particularly advantageously found that insonation of the cancer tissue with intensities of as low as 4.6 W/cm² were effective in selectively killing cancer cells. Thus, in some embodiments the present method is applicable when the US frequency is between 20 kHz and 35 kHz. In the above and other embodiments, the present method is applicable when the intensity is between about 0.3 and 5.5 W/cm² and the frequency is 20 kHz. According to various specific embodiments of the present disclosure, ultrasound is applied at an intensity of up to 5 W/cm² (e.g., between 4 and 5 W/cm²) at a frequency of 20 kHz.

In certain embodiments, the ultrasound can be administered continuously. The time interval required for US irradiation can be determined based on parameters known to a skilled artisan, while considering, inter alia, the parameters mentioned herein.

Another advantage of the methods and devices provided herein is that the hyper-proliferative cells can be effectively treated in short time periods. According to one embodiment, the method described herein is characterized in applying the ultrasound at an application time ranging from about 5 seconds to 30 minutes, e.g., between 5 seconds and 25 minutes, between 5 seconds and 20 minutes, between 5 seconds and 15 minutes, between 5 seconds and 10 minutes, between 5 seconds and 5 minutes or between 10 seconds and 10 minutes. These alternative time intervals refer to each separate treatment, and may be repeated as part of a treatment series. Preferably, the application time of each separate application is up to 20 minutes, more preferably, about 15 seconds.

In other embodiments, the ultrasound can be administered intermittently, using ON/OFF cycles. The effective ON/OFF cycle times may be determined depending, for example on the volume of tumor, type of tumor, and other relevant variables.

As detailed in the appended Examples below, the ability of low frequency ultrasound to selectively inhibit brain tumor tissue growth in vivo was established for two regimens: to be applied once (also termed herein “US1X”) or to be applied twice, with a 24-hour interval between applications (also termed herein “US2X”). The experimental results, as shown below, demonstrated rapid inhibition of tumor growth. In the course of the regimen including two US applications (US2X), the tumor tissue was completely destroyed, and an extensive immune response was observed. Importantly, the treatment was highly selective, keeping the healthy tissue surrounding the tumor unharmed.

Therefore, in some embodiments, the method according to the present disclosure comprises applying ultrasound at least once, or multiple times, at a regimen frequency of between once or twice daily to once weekly, during a period of between one to four weeks. In particular embodiments of the method of treatment as herein defined, ultrasound regimen of once or twice daily or once weekly during a period of between one to four weeks may be repeated, e.g. after a treatment intermission of about a month, two months, or more.

As shown in the appended Examples, the US effect on the tumors was affected by the dimensions of the ultrasound probe (transducer) such that a maximal effect was obtained for tumors having volumes (or dimensions) which were comparable to the dimensions of the transducer. Therefore, when applying US, the transducer (referred to interchangeably herein as “probe”, “tip” or “device”) should be selected such that their structure and dimensions are suitable to the estimated dimensions of the tumors being treated. Methods for estimating the tumor volume and dimensions are known in the art, for example, computed tomography (CT) or magnetic resonance imaging (MRI). In certain specific embodiments, the method according to the present disclosure is wherein ultrasound is applied essentially at an a perpendicular axis to said organ or tissue, e.g., at an angle of between about −60° and about 60°, preferably at an angle of between about-30° and about 30° relative to a perpendicular axis to said organ or tissue. US may be performed from several perspective, e.g., to maximize or enable the exposure of a tumor, e.g., a tumor that is larger than the transducer used.

The irradiating step as herein defined is a direct US insonation, i.e., it is performed by placing the ultrasound transducer in the proximity of said tissue or organ, namely, in a position that is close to the affected area being treated. By the term “proximity” as used herein it is referred to between about 1 mm to about 5 cm.

As shown by the present inventors, beneficial US treatment was obtained when the US was administered by irradiating the tumor intracranially, i.e., inside the skull. In other words, the US was directly applied to the tumor-bearing area in an invasive manner, after an excision is made in the bone and tissue. Therefore, the irradiating step may be performed by placing the ultrasound transducer within a cavity formed by surgery in the vicinity of said organ or tissue. The cavity may be formed by surgery, as known to a skilled physician.

The methods provided herein can be used as a monotherapy or in conjunction with other therapies (also referred to herein as adjuvant therapy), for example, pharmacotherapy such as, without being limited to, photosensitizers, sonosensitizers, chemotherapeutic and immunotherapeutic agents; and or in conjunction with other therapies such as, without being limited to, anticancer radiotherapy or surgery. Such combinations may offer significant advantages, including synergistic activity, in therapy. In addition, such combinations may provide an improved quality of life compared to the quality of life the same patient would experience if they received only the chemotherapeutic agent as therapy. For example, the combined therapy with the low frequency ultrasound described herein may lower the dose of chemotherapeutic agents needed, thereby lessening the side-effects associated with high-dose chemotherapeutic agents. The combination may also cause reduced tumor burden and the associated adverse events, such as pain, organ dysfunction, weight loss, etc.

In some particular embodiments, the method according to the present disclosure is an adjuvant therapy to surgery of the organ or tissue affected by cancer.

In other particular embodiments, the method according to the present disclosure may be applied for eliminating non-resected margins of said cancer in said organ or said tissue, post-surgery (after a surgery is performed for resecting the tumor in an organ or a tissue). This particularly applies to cases in which the resection is incomplete and/or is merely a de-bulking procedure. US applied according to the present disclosure may then be applicable to irradiate/eliminate the margins that are otherwise non-respectable, and thereby prevent or decrease the prevalence of further metastasis thereof. For example, for applying this method in cases of ovarian, mammary, or pancreatic cancers, safety experiments to the surrounding tissues may be performed, as exemplified in Examples, in order to determine the required US conditions. Alternatively, lower intensity US below the LF-MI of 3 and TI below 5 may be used.

As used herein, the term “treating” or “treatment”, as known in the art, refers to an approach for obtaining beneficial or desired results, including clinical results. These include, but are not limited to, curing, alleviation or amelioration of one or more symptoms or conditions, diminishment of extent of disease, stabilizing (i.e., preventing or delaying the worsening) the state of disease (e.g., maintaining a patient in remission), preventing or reducing spread of disease, delay or slowing of disease progression, amelioration or palliation of the disease state, diminishment of the reoccurrence of disease, and remission (whether partial or complete), whether detectable or undetectable. The term “treating” and “treatment” can also mean prolonging survival as compared to expected survival based on existing epidemiological data on the date of “treatment”, if not receiving treatment. The term “treating” also includes inhibiting future onset or development of the disease.

As used herein, the term “subject” includes all members of the animal kingdom including mammals, and more preferably refers to humans. Optionally, the term “subject” includes mammals that have been diagnosed with cancer or are in remission therefrom, or mammals that have been diagnosed with injury, e.g., head injury.

By the terms “disease” or “condition” it is referred to any disease, disorder, condition or to any pathological or undesired condition, state, or syndrome, or to any physical, morphological or physiological abnormality which may benefit from the method as herein defined. In particular, the terms “disease” or “condition” as used herein, encompass diseases which are associated with an abnormally proliferating cell population, and in particular diseases which are associated with an abnormally hyper-proliferating cell population, such as tumors (pre-malignant or malignant) and/or cancers. Preferably, the cancers may be selected from diseases and disorders classified in any one of subclasses 02A20 to 02F9Z, of the 11th revision of World Health Organization International Classification of Diseases. In some particular embodiments, the disease ort disorder may be selected from the group consisting of a sarcoma, a carcinoma, a lymphoma, a cancer of the breast, of an ovary, of the lung, of the prostate, of the colon, of the skin, and of the bladder. The cancer may be a primary cancer, or an ectopic metastasis of a cancer of different origin.

As detailed herein, the experimental results show high selectivity accompanied with high efficacy when US monotherapy was used, namely when US was the primary treatment tool for treating malignant glioma in vivo. Low frequency US applied in conditions which were shown to be safe to healthy tissue induced rapid inhibition of tumor growth. In addition, the inventors have demonstrated that insonation specifically damaged the tumoral tissue, resulting in its necrosis, which is confined by gliotic reaction with time. When US was applied twice, the glial reaction was more progressed, and the gliosis was more extensive, all the tumor tissue was destroyed and was being cleared. One of the most important observations was the minimal neurotoxicity of the US treatment, while the tumor tissue was destroyed by the US treatment, the healthy tissue surrounding the tumor tissue was left unharmed, demonstrating that the tumor was eliminated in a selective manner.

By way of example, cancer includes brain cancers (also referred to herein as brain tumors), e.g., primary brain tumors as well as metastatic tumors that metastasize to the brain from other areas in the body. Determining the proliferative state of cells and diagnosis of hyperproliferating cells and a disease or disorder associated therewith are performed by a skilled physician. In particular embodiments, the method of the present disclosure is applicable to glioma, but could also be applied to other solid cancers, including but not limited to sarcomas, carcinomas, lymphomas, and cancers in the breast, ovary, lung, prostate, colon, skin, and bladder.

In particular embodiments, the method according to the present disclosure is suitable for treating a brain tumor wherein the irradiating step is performed by placing the ultrasound transducer within a cavity formed by brain surgery. In some further embodiments, irradiating step is performed by placing the ultrasound transducer in a proximity of an affected organ or tissue during a surgery.

In view of the foregoing, in a preferred aspect, the disease or a disorder is a primary brain cancer. The primary brain cancer may be selected from diseases or disorders classified in a subclass 02A00, 02A01, 02A02, or 02A0Z, of the 11th revision of World Health Organization International Classification of Diseases. Specifically, in some particular embodiments, the primary brain cancers may be a glioma, a gliosarcoma, an astroblastoma, an astrocytoma, a neurocytoma, a meningioma, a medulloblastoma, or a neuronal-glial tumor. Thus, by a preferred aspect thereof the present disclosure provides a method of treating a subject suffering from a primary cancer of a brain, said method comprising directly irradiating the cancerous lesion or non-resected tumor margins in a post-operative cavity with ultrasound having a frequency of between 20 and 200 kHz, at an ultrasound intensity of between 1 and 50 W/cm², and wherein said ultrasound intensity is characterized by a thermal index (TI) value of between 0.1 than 5 and by a low-frequency mechanical index (LFMI) value between 0.15 than 3. Therefore, in some preferred embodiments, the direct irradiating is performed by placing the ultrasound transducer onto the cancerous lesion or into the post-operative cavity, and applying the ultrasound. Preferably, the ultrasound is administered as adjunctive treatment following resection, to insonate the possible unresected margins. Alternatively, the ultrasound may be administered prior to resection, in neo-adjuvant manner, to limit the surgery in scope and/or in the invasiveness. Despite the apparent need for repetitive interventions, the ultrasound may be administered more than once, e.g., twice or more times, to achieve the desired insonation volume and the therapeutic results.

Preferably, the method may be performed with the ultrasound frequency being between about 20 kHz and 35 kHz, e.g., between 18-22 and 35 kHz. Further preferably, the intensity may be between about 1 and 5.5 W/cm², particularly if the frequency is between about 20 and 22 kHz. As detailed above for more general embodiments, the ultrasound may be applied at least once for a time interval of between 5 seconds and 30 minutes, depending on the required intensity and the available frequency.

Generally, the frequency and intensity parameters are selected such that the thermal index value of the ultrasound be selectable from the group consisting of between 0.1 and 5.0, between 0.1 and 4.5, between 0.1 and 4.0, between 0.1 and 3.5, between 0.1 and 3.0, between 0.1 and 2.5, between 0.1 and 2.0, between 0.1 and 1.5, and between 0.1 and 1.0, and the low-frequency mechanical index value be selectable from the group consisting of between 0.15 and 3.0, between 0.15 and 2.8, between 0.15 and 2.6, between 0.15 and 2.5, between 0.15 and 2.4, between 0.15 and 2.2, between 0.15 and 2.0, and between 0.15 and 1.9.

As detailed above, the present disclosure also provides treatment by US of brain tissue injury. The experimental results presented in the Examples below show, inter alia, decrease in the lesion area and the necrotic area in US-treated groups as compared to the control group. Thus, the terms “disease” or “condition” further include injury afflicted to an organ or a tissue in a subject, such as but not limited to head injury, e.g., traumatic head injury, injury caused by stroke or injury caused by surgery, e.g., brain injury. Additionally, the “disease” or “condition” encompass inflammatory conditions, in particular brain inflammatory conditions, such as caused, e.g., by an injury or hemorrhage.

In other words, the present disclosure further provides a method of treating a subject suffering from a brain injury, said method comprising directly irradiating an area of said brain injury with ultrasound having a frequency of between 20 and 200 kHz, at an ultrasound intensity of between 0.3 and 50 W/cm², wherein said ultrasound intensity is characterized by a thermal index (TI) value lower than 1.0 and a low-frequency mechanical index (MI) value lower than 1.9. However, as discussed above the thermal index of the US treatment may be kept below 5.0, and the low-frequency mechanical index may be kept below 3.0.

As known in the art, injuries to the central nervous system can present in myriad ways. This can be seen in a variety of settings; global insults seen in traumatic brain injuries, space occupying lesions such as tumors and hemorrhagic strokes, to progressive neurodegenerative disorders such as Alzheimer's and Parkinson's disease. In some particular embodiments, brain injury results from traumatic brain injury.

As known in the art, medical intervention after brain injury may be confined to a defined timeframe. In particular embodiments, treatment by US of brain tissue injury according to the present disclosure is applied between 10 minutes and 48 hours post injury.

Various features according to the present disclosure, as described herein in reference to one aspect thereof, are applicable mutatis mutandis to other disclosed aspects according to the teachings herein inasmuch there are no contradictory more specific disclosure regarding these specific aspects. It must also be noted that, as used in this specification and the appended claims:

All scientific and technical terms used herein have meanings commonly used in the art unless otherwise specified. All scientific and patent publications mentioned in the present disclosure are incorporated herein by reference. The definitions provided herein are to facilitate understanding of certain terms used frequently herein and are not meant to limit the scope of the present disclosure. The term “about”, “c.a.”, “~”, and like, as used interchangeably herein, as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range, preferably as used herein the term “about” refers to +10%. The terms “comprises”, “comprising”, “includes”, “including”, “having” and their conjugates mean “including but not limited to”. This term encompasses the terms “consisting of” and “consisting essentially of”, which have their narrower meaning as known in the art, thus an embodiment described as comprising something also discloses embodiments consisting essentially of same and consisting exclusively of same. Various embodiments and aspects of the present invention as delineated hereinabove and as claimed in the claims section below find experimental support in the following examples. The following examples are representative of techniques employed by the present Inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention. As used herein, a phrase in the form “A and/or B” means a selection from the group consisting of (A), (B) or (A and B), and as used herein, a phrase in the form “at least one of A, B, and C” means a selection from the group consisting of (A), (B), (C), (A and B), (A and C), (B and C) or (A, and B, and C), and further combinations are envisaged for the lists comprising larger number of terms. It is appreciated that certain features of the invention, which are, for brevity, described in the context of separate embodiments, may also be provided in combination in a single embodiment, unless technically infeasible. Conversely, combinations of various features of the invention, which are, for clarity and demonstration, described in the context of a single embodiment, may also be provided separately or in any suitable sub-combination, or as reasonable to the skilled artisan, suitable, and operative. Certain features described in the context of various embodiments, including preferred features, are not to be considered essential features of those embodiments, unless the embodiment is inoperative without those elements. The singular forms “a”, “an” and “the” include plural referents unless the content clearly dictates otherwise.

As used herein below, the abbreviations indicate as follows: GBM, glioblastoma multiforme; FDA, Food and Drug Administration; DMEM, Dulbecco's Modified Eagle Medium; RGS, Rat Grimace Scale; H&E, hematoxylin/eosin; TI: thermal index; TIs, thermal index for soft tissue; MI, mechanical index; MRI, Magnetic Resonance Imaging; US, ultrasound; US1X, US application once; US2X, US application twice with a 24 hour gap; GMCs, multinucleated giant cells; DAMPs, danger associated molecular patterns; MTT, 3-(4,5-dimethylthiazol-2-yl)-2,5-diphenyltetrazolium bromide.

### EXAMPLES

Reference is now made to the following examples, which together with the above descriptions illustrate some embodiments of the invention in a non-limiting fashion.

### Methods

### Ultrasound Dosimetry

As described in the general description section above, the ultrasound parameters that may be important to consider in the US treatment include the thermal index and the mechanical index of the ultrasound. The thermal index (TI) is a measure of the US thermal bio-effect, used in order to estimate the expected temperature rise due to acoustic energy exposure for assuring no thermal damage is caused. For soft tissues, the TI (TIs) is defined as the ratio of the emitted acoustic power to the power required to raise the tissue's temperature by 1 °C. According to the FDA safety guidelines, TI should be lower than the value of one (FDA, Information for Manufacturers Seeking Marketing Clearance of Diagnostic Ultrasound Systems and Transducers, Ultrasound. (2008) 1-64). The TIs was calculated by equation 1 below:

$${TI}_{s} = \frac{{Wf}_{c}}{210} < 1 \qquad ( 1 )$$

where, W is the output power in mW, and f_{c} is the center frequency in MHz (W. D. O'Brien and D. Scott Ellis, IEEE Trans. Ultrason. Ferroelectr. Freq. Control. 46 (1999) 1459-1476. https://doi.org/10.1109/58.808870).

The mechanical index (MI) is a measure of the acoustic output and is an indicator for the probability of US mechanical (i.e. non-thermal) bioeffects. As discussed above, for the purpose of the present disclosure, the thermal index of the US treatment may be kept below 5.0, and the mechanical index may be kept below 5.0, e.g., below 3.0. For the low-frequency US, low-frequency mechanical index (LF-MI) can be calculated using an adjusted index (F. Ahmadi et al., Prog. Biophys. Mol. Biol., 108 (2012) 119-138, https://doi.org/10.1016/j.pbiomolbio.2012.01.004) by equation 3:

$$MI = \frac{P - {P}_{0}}{\sqrt{{f}_{c}}} \leq 1.9 \qquad ( 3 )$$

where, P is the pressure amplitude in MPa, P₀ is the ambient pressure in MPa, f_{c} is the center frequency in MHz, which is used herein and in particular below.

### Example 1

### Reduction in Glioma Cells Viability In Vitro by Low Frequency US

C6 rat glioma cells were grown in cell culture media DMEM (Dulbecco's Modified Eagle Medium) containing 10% (v/v) fetal bovine serum, 1% (v/v) Penicillin/Streptomycin (pen-strep), and 1% (v/v) L-glutamine on uncoated flasks at 37 °C under 5% CO₂ atmosphere. Cells were passaged every 72-96 hours. Materials were purchased from Biological Industries (Kibbutz Beit-Haemek, Israel).

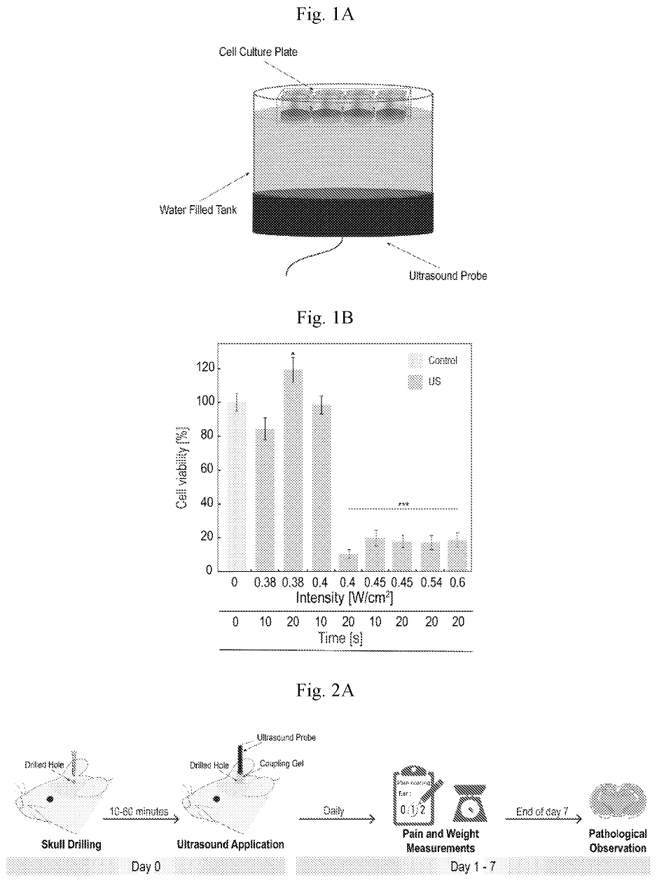

To test the effect of US on cell growth, cells were seeded at a density of 100,000 cells/mL and grown to ~70% confluence in a 24-well plate. The plate was placed on top of a water-filled reservoir, 3 cm above an ultrasonic 13 cm microplate horn (20 kHz Misonix-ultrasonic liquid processor S4000-010, max intensity 600 W) as schematically shown in FIG. 1A. In the Figure, the cell plate (denoted as “Cell Culture Plate”) was placed on top of a water-filled tank (denoted as “Water-Filled Tank”) above the ultrasonic microplate probe (denoted as “Ultrasonic Probe”). US was applied at intensities of between 0.38 W/cm² to 0.6 W/cm² in a continuous mode for durations of 10 or 20 seconds. After US application, cells were incubated for 1 hour and then an MTT (Thiazolyl Blue Tetrazolium Blue) viability assay was performed according to the manufacturer's instructions. Briefly, media was replaced with starvation medium (250 μL of 5% (V/V) serum, 1% (V/V) L-glutamine, and 1% (V/V) pen-strep in DMEM). MTT reagent (25 μL, Sigma-Aldrich Inc.) was added and the plate was mixed gently. After 2 hours of incubation at 37 °C, 500 μL solution of isopropanol (Sigma-Aldrich Inc.) containing 0.04N HCl (Sigma-Aldrich Inc.) was added and the plate was wrapped in aluminum foil and placed in the sterile hood, overnight. Absorbance was measured 24 hours later, at 570 nm wavelength using an ELISA reader (Bio TEK instruments, ELX 808). Results were expressed as viability percentage relative to untreated cells. Statistical analysis of variance and Student's t test were applied. For all tests, a value of p<0.05 was interpreted as significant.

The results of the evaluation indicate as follows. For example, as shown in FIG. 1B, US intensities of 0.38 W/cm² for 10 or 20 seconds (denoted in the lower horizontal axis as “Time [s]”), and intensity of 0.4 W/cm² for 10 seconds (denoted in the upper horizontal axis as “Intensity [W/cm²]”), exhibited high cell viability of more than 80%. In the Figure, MTT assay was performed 1 hour after insonation. Bars represent mean #SEM based on 3 individual experiments; *p<0.05, **p<0.01, ***<0.001 in comparison to the control by t-Test of two samples assuming unequal variance. The lighter hue bar on the left side represents control cells, and the darker-hue bars represent the viability of the insonated cells. As can be readily seen, intensity of 0.4 W/cm² for 20 seconds and higher intensities, exhibited very low cell viability of under 20% (P value <0.001). The reduction in cell viability at longer duration and higher intensities indicates that 20 kHz low frequency US inhibited cell proliferation and induced toxic effect on the cells. Without wishing to be bound by any theory, the underlying mechanism may be induction of sonoporation (the disturbance and formation of pores in the cellular membrane) by US, in this case the formation of irreversible pores leading to spontaneous cell death. In summary, this experiment shows that glioma cells are highly sensitive to 20 kHz low frequency US, which means that this frequency has therapeutic potential.

### Example 2

### Safety of US Conditions In Vivo

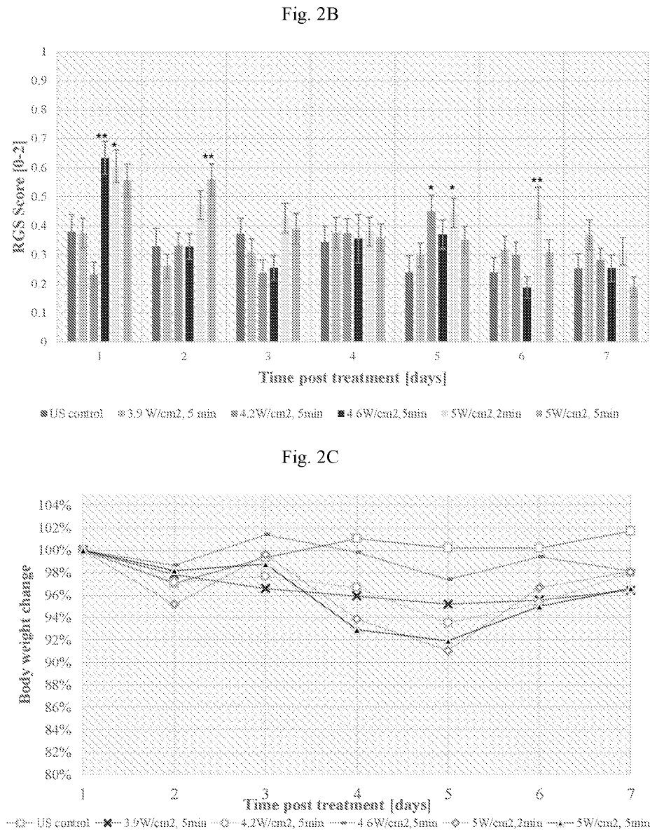

In order to evaluate the safe conditions for US operation, the following safety assays were conducted which included a surgical procedure and insonation, followed by safety examinations during the following 7 days for assessing pain (through changes in facial expressions) and weight change. The scheme of the safety assays is graphically presented in FIG. 2A-briefly, the skull was drilled to make an opening therein (denoted as “Skull Drilling” and “Drilled Hole”, respectively, on the artistic representation of the test animal head) and US at intensities of 3.9, 4.2, 4.6, 5 W/cm² for 5 minutes and 5 W/cm² for 2 minutes was applied via the drilled hole using the probe and coupling gel (denoted as “Ultrasound Application”, “Ultrasound Probe”, and “Coupling Gel”, respectively, on the second representation of an animal head from the left). The bar below on the left side and the arrow between the artistic representations of the animal heads indicate the timeline (denoted as “Day 0” on the time bar, and as “10-60 minutes” above the arrow). Pain and weight were measured (as denoted by artistic representation of a tablet and a pen, and scales, and by label “Pain and Weight Measurements”) daily (as denoted by the second arrow from the left and the label “Daily”) for 7 days (as denoted by the right bottom bar and the label “Day 1-7”), and on day 7 the brains were examined by pathological observation (as denoted by the rightmost arrow with the label “End of Day 7”, the artistic representation of a brain cross-section, and the label “Pathological Observation”).

Female Fischer 344 rats, weighing 125-175 g each (Harlan Bioproducts, Indiana, IN) were housed in standard facilities and provided with ad libitum access to food and water. The policies and guidelines of the Johns Hopkins University Animal Care and Use Committee were followed throughout the study under an Animal Care and Use Committee-approved protocol.

Rats were anesthetized with a 0.4 mL/body weight intraperitoneal injection of a stock solution containing ketamine HCl (75 mg/Kg, Covetrus; xylazine (0.75 mg/Kg, Covetrus). Animals were given pre-emptive analgesia of buprenorphine at a dose of 0.01-0.05 mg/kg, SC, prior to surgery. The head was shaved and prepared with alcohol and Prepodyne™ solution (DeLaval Inc.) and a 1-cm midline scalp incision was made exposing the sagittal and coronal sutures. A 3 mm burr hole was made in the skull, 5 mm posterior and 3 mm to the right of the bregma, using an electric drill. Post-operative care included daily observations, removal of surgical clips and analgesia was provided as needed. All surgical procedures were performed using standard sterile surgical techniques.

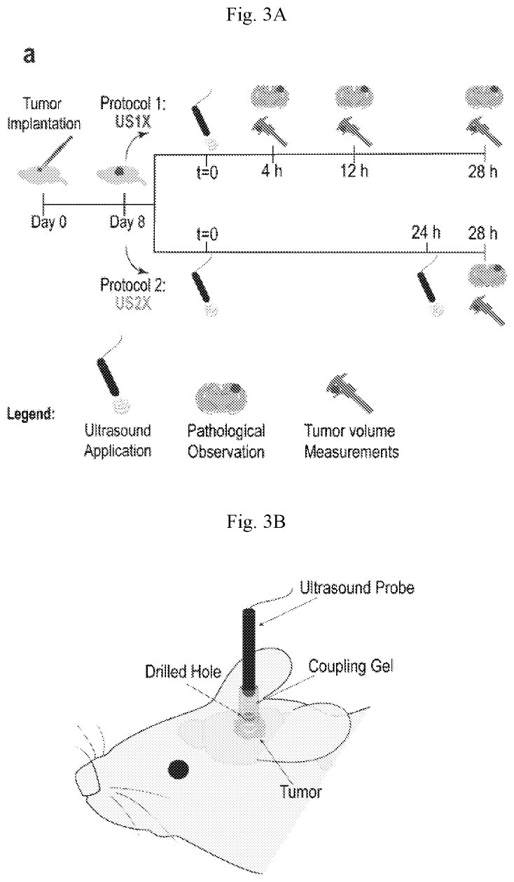

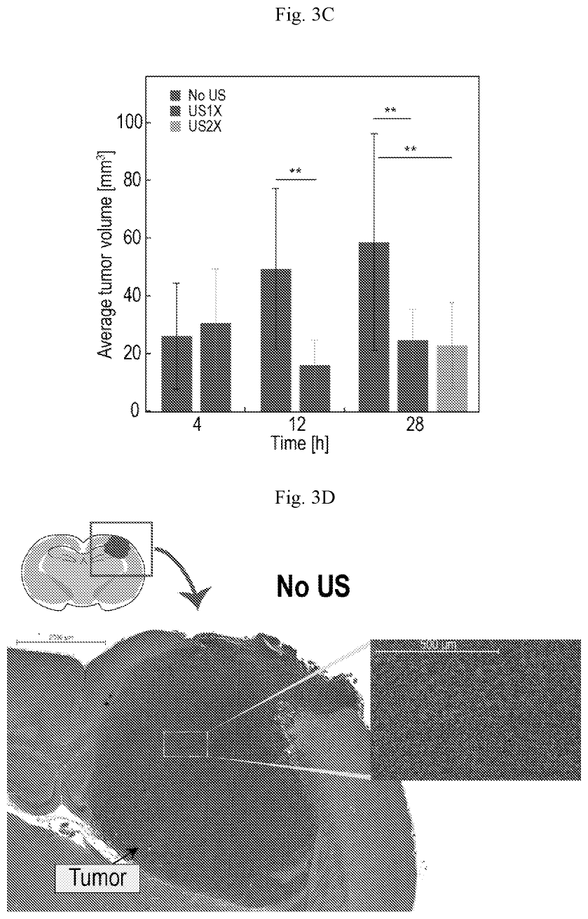

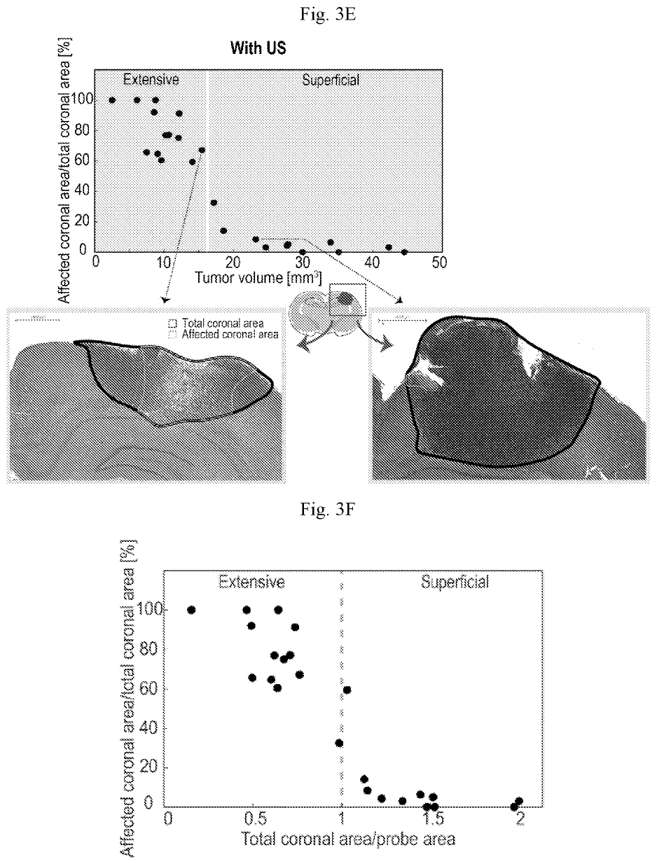

For insonation, anesthetized animals (n=6) were placed in a custom-made head-stabilizing stand on a vertically displaceable platform to control the height/distance of the US probe. On top of the head, a cylinder (having a diameter of 1.5 cm) was placed. The US probe was inserted into the cylinder right on top of the burr hole, and the probe height was fixed at 2 mm above the drilled hole in the skull (as schematically shown in FIG. 3B). Next, the cylinder was filled with US gel and US was applied. During US application, the gel was constantly (manually) replaced, such that the probe was always immersed in the gel in order to keep the gel temperature and other properties constant. US at a frequency of 20 kHz (Q125 Sonicator, Qsonica L.L.C, Newtown, CT, USA) with a probe tip having a diameter of 3.2 mm was used. US was applied in a continuous mode, 10-60 minutes after the skull was drilled and under full anesthesia. The applied US conditions were 3.9, 4.2, 4.6, 5 W/cm² for 5 minutes and 5 W/cm² for 2 minutes. The skin was then closed with skin glue (3M Vetbond™ tissue adhesive). As a control, the same surgical protocol was followed, apart from the US application.

For safety evaluation, rats were monitored daily for 7 days for weight change and pain.

Rats were monitored for quantification of pain using rat grimace scales (RGS) according to a published method (S. G. Sotocina, et al., Mol. Pain. 7 (2011) 1744-8069-7-55. https://doi.org/10.1186/1744-8069-7-55). Accordingly, rats were monitored daily, for seven (7) days, for pain related facial “action units” defined by RGS. The four action units are orbital tightening, nose/cheek flattening, ear changes, and whisker change. Scoring was performed according to the description and guiding figures described in the above published method. The scoring range was 0-2, where score of “0” indicated that the action unit was absent, score of “1” indicated a moderate appearance of the action unit, and score of “2” indicated an obvious appearance of the action unit. Monitoring was performed by digital video; rats were placed separately in a cubical transparent box on a table, and were filmed via digital video camera (Canon HD Camcorder VIXIA HV40) for 20-30 minutes while making sure a clear head frames were acquired. Next, facial images were acquired manually from the digital videos. Pinnacle Studio™ 14 video editing software was used to create short videos of the clear head frames of each rat and then Irfan View software was used to extract images from these videos. For RGS scoring, a single most suitable image was manually selected. RGS scoring was performed by presenting randomly organized non-labeled images to an assessment panel consisting of six trained postdoctoral, graduate or undergraduate students for blinded images scoring. Pain was quantified by averaging the action unit scores across coders. The variability within each group was described by standard error of mean (SEM). Statistical analysis was performed using XLSTAT software v. 2019 (www.xlstat.com). Group differences were performed by one-way ANOVA followed by Dunnett's case comparison post-hoc test. For all tests, a value of p<0.05 was interpreted as significant. Examining the average spontaneous pain through facial expression scoring, FIG. 2B show that the pain values were relatively low; the vertical axis represents the RGS score (denoted as “RGS Score (0-2)”), and along the horizontal axis the bars that represent RGS score mean±SEM RGS for various insonation conditions as function of elapsed time (denoted as “Time post treatment [days]”) are presented. Further in the Figure, *P<0.05, **P<0.01, ***P<0.001 compared to the control by Dunnett's case comparison post hoc test (one-way). All values were in the range of 0 to 1, meaning that the highest score was moderate appearance of the action unit. In the control group the score values were around 0.3 at all the time points. The same pattern can be seen for 3.9 W/cm². In the group of 4.2 W/cm², all the score values were as the control except for day 5 which the value was higher and statistically different. In the group of 4.6 W/cm², the score in the first day was ~0.6, and in the next days the values were as the control. In the group of 5 W/cm² for 2 min, score values higher than the control were observed on days 1, 2, 5, and 6. In the group of 5 W/cm² for 5 min, score values higher than the control were observed on days 1, and 2. Overall, the highest values were observed in the first 2 days, and on day 5, meaning that the main pain effect was in these days. The highest pain effect (more days with values higher than the control) were found for the groups of 5 W/cm², even though all values were found to be on the scale of moderate pain and less.

In addition, as can be seen in FIG. 2C, in all the groups tested, relatively stable weight with no drastic changes was observed. In the graph, bars represent mean±SDM. In the control group the body weight was slightly decreased on day 2 (~3%) and then gradually increased reaching 2% of increase on day 7. In the groups of 3.9 W/cm² and 4.6 W/cm² weight was relatively stable, and in the group 4.2 W/cm² the same pattern was observed except for day 5 were 6% of decrease is observed. For the groups of 5 W/cm² a decrease in weight of 8-9% was observed on days 4 and 5, this correlates with higher pain values observed on days 5 and 6. Most of the changes were observed on days 4 and 5 and are in the range of ~10% from the initial weight. Pain measurements and body weight results values were found to be low, indicating the tested US protocols are safe for short term of the duration of the experiment, with the most notable effect seen in the first and fifth days, for high US intensities groups.

Histopathological evaluation of safety and biocompatibility of ultrasound application were assessed at tissue level seven days after the surgical intervention. For each group, after pain evaluation was completed, the brains were carefully removed, fixed using 4% paraformaldehyde, embedded in paraffin, and sliced with a microtome to obtain coronal sections of 10 μm. Sections were than stained with hematoxylin/eosin (H&E) for histological observation to evaluate tissue damage. Brain tissue was examined in a blinded manner. The histopathological evaluation of control rat brains found common features at the drilling site. Superficial injury was presented only in grey matter. Histological evaluations of US-treated rat brains were similar to the control group.

As can be seen in Table 1, the TI of all intensities examined are within the safe limitations defined by the FDA. The LF-MI of the intensity of 5 W/cm², slightly exceeded the upper limit value defined by the FDA, and all other intensities are within the safe limitations defined by the FDA.

TABLE 1
Calculation of TI, LF-MI for ultrasound application at different
intensities examined in vivo for 5 mm probe distance.

| Intensity (W/cm²) | TI | LF-MI |
|---|---|---|
| 5.0 | 0.74 | 2 |
| 4.6 | 0.68 | 1.9 |
| 4.2 | 0.63 | 1.8 |
| 3.9 | 0.58 | 1.7 |

From the overall investigation of safety, it may be concluded that US irradiation of 5 W/cm² for 2 minutes and 5 minutes were found to have toxicity mainly observed by histopathology features and by LF-MI higher than the upper limit defined by the FDA, and US irradiation of 4.6 W/cm² and below were found to be safe under the experimental conditions at all methods examined.

### Example 3

### US Inhibits Tumor Growth and Leads to Tumor Elimination In Vivo

Based on the results obtained in the safety measurements above, the US intensity of 4.6 W/cm² for a period of 5 minutes was found to be safe for healthy brain tissue in all methods examined, and was therefore chosen for therapeutic application, specifically, for treating brain tumors.

Tumor growth with and without US application was evaluated by measuring the tumor volume as a function of time. In each one of the groups, 8-14 rats per time point were evaluated. For volume measurements, a coronal section was performed at the center of the tumor, the maximum height and length of the tumor were measured, and volume was calculated using a half spheroid approximation according to equation 3 below:

$$V = 0.5 \cdot \frac{4 π}{3} {a}^{2} c \qquad ( 3 )$$

where, a is half the total tumor length at the transverse plane, and c is the tumor depth in mm. This calculation was verified by measuring the volume of several brains using Magnetic Resonance Imaging (MRI), by scanning the brains before sectioning and summing all T2-weighted images of hyperintensity lesion areas. The MRI volume calculations were found to correlate, with less than 10% bias, with the volume calculated using the half spheroid approximation. Statistical analysis of variance was applied in order to determine if there is a change in the tumor volume in time. For all tests, a value of p<0.05 was interpreted as significant. All data are reported as the mean±SD.

An in vivo study was conducted to examine the effect of US irradiation of 4.6 W/cm² for 5 minutes on an intracranial 9 L gliosarcoma tumor mass in terms of volume growth and structural changes. Two protocols of US were used, the first one consisted of application of US once (termed herein “US1X”) and the second one consisted of application of US twice with a gap of 24 hours in between (termed herein “US2X”), as schematically shown in FIG. 3A and FIG. 3B. In the FIG. 3A, two US protocols were examined on day 8 after tumor implantation (denoted as “Day 8”): US application once (denoted as “Protocol 1: US1X”,) and US application twice (denoted as “Protocol 2: US2X”,) as illustrated; one control protocol, untreated group, is not shown. The artistic renditions in the Figure are explained in the legend (denoted as “Legend:”) below the scheme, as follows: the artistic representation of ultrasound probe is denoted as “Ultrasound application”, the artistic representation of a brain cross-section with a tumor is denoted as “Pathological Observation”, and an artistic representation of a caliper measuring a mass is denoted as “Tumor volume Measurement”. In the Figure, the tumor implantation is shown on day 0 on the leftmost rendition of an animal, denoted as “Tumor Implantation” above the rendition and “Day 0” below same. The time points corresponding to time zero (denoted as “t=0”), 4 hours, 12 hours, 24 hours, and 28 hours (denoted as “4 h”, “12 h”, “24 h”, and “28 h”, respectively) are shown along the timeline axes of the corresponding protocols. In FIG. 3B, an artistic rendition of the US probe (denoted as “Ultrasound Probe”) is shown immersed in a gel-filled cylinder (denoted as “Coupling Gel”), 2 mm above the drilled hole (denoted as “Drilled Hole”) in the skull of a tumor bearing rat (denoted as “Tumor”). For efficacy evaluation, tumor growth was examined by measuring the tumor volume at different time points post US application, and comparing it to the untreated group.

Animals were handled as in the Example 2 above, following the same intracranial drilling procedure. Rats were intracranially implanted with 9 L gliosarcoma, which was maintained and passaged every 2-3 weeks in the flank of donor Fisher 344 rats (Yawen Li et al., In vivo delivery of BCNU from a MEMS device to a tumor model, Journal of Controlled Release, Volume 106, Issues 1-2, 18 Aug. 2005, Pages 138-145). For surgical intracranial implantation of the tumors, the tumor was removed from the carrier animal, cut into ca. 2 mm³ pieces, and placed in sterile 0.9% saline on ice. Through the burr hole, which was made in the skull and under microscopic magnification, a dural opening was made. A small area of cortex and white matter was resected. Once hemostasis was achieved, a single tumor piece (2 mm³) was placed into the resection cavity. The skin was then closed with surgical staples. All surgical procedures were performed using standard sterile surgical techniques.

Eight days after tumor implantation, the incision was opened under anesthesia and the burr hole was located. Tumor bearing rats were divided into groups (8-14 rats per group) and were placed in a custom-made stand as described above. US was applied in continuous mode for 5 minutes at an intensity of 4.6 W/cm². The groups included: (1) Control group, undergoing no US treatment; (2) US applied once group, undergoing a protocol of one US application (also termed herein “US1X”); (3) US applied twice group, undergoing a protocol of two US applications, with a gap of 24 hours between applications (also termed herein “US2X”). After each experimental treatment, the incision was stapled, and the animals were allowed to awake and recover. For groups 1 and 2, the tumors were examined at 4, 12 or 28 hours post US application (for the control group, time was counted from the beginning of the experimental treatment applied to the additional groups). For group 3, the tumors were evaluated only 4 hours after the second US application (i.e., 28 hours after the first US application). At each time point, rats were euthanized, the brains were carefully removed and placed in 4% paraformaldehyde for at least 24 hours.

In each one of the groups, six rats per time point were evaluated histologically. Brains were embedded in paraffin to obtain coronal sections of 10 μm, then stained with H&E staining for histological observation to evaluate the tumor structural changes. Brain tissue was examined in a blinded manner. The morphologic terminology was based on the state-of the art harmonized terminology (A. E. Bradley, et al., Toxicol. Pathol. 48 (2020) 827-844. https://doi.org/10.1177/0192623320951154). In addition, the affected area in the tumor, which was evaluated histologically, was measured using ImageJ software (C. T. Rueden, et al., BMC Bioinformatics. 18 (2017) 529. https://doi.org/10.1186/s12859-017-1934-z) and compared to the total tumor area at each section. These ratio values were than plotted as a function of tumor volumes.

As shown in FIG. 3C, in the untreated group (indicated “No US”), a rapid growth was observed. In the Figure, the average tumor volumes (denoted by “Average Tumor Volume [mm³]” label on the ordinate axis) are plotted as bars as function of time (denoted by “Time [h]” label on the abscissa axis). Average tumor volume is shown as a function of time post-US treatment for the different experimental groups (8≤n≤14), control group (denotes as “No US”), group treated with US1X protocol (denoted as “US1X”), and group with US2X protocol (denoted as “US2X”); *p<0.05, **p<0.01, ***<0.001 in comparison to the control group (t=0) by t-Test of two samples assuming unequal variance. Average tumor volume was doubled between the time points of 4 hours and 12 hours and increased by ~1.2 folds on average between 12 hours to 28 hours. It is important to note that the experiment was done 8 days after tumor inoculation, which is a progressed stage of the tumor growth, and therefore rapid increase in tumor growth in the control group was expected. The untreated group and the group that was treated with US1X, had similar tumor volumes at 4 hours. However, by 12 hours, the volume of the US1X group had decreased 3-folds (P value <0.01) compared to the untreated group, and 2-folds when compared to the average volume of the two groups back at 4 hours time-point. A similar observation was found at 28 hours, with a 1.2-fold significant decrease (P value <0.01) in the group that received US1X compared to the untreated group. The average tumor volume of the US2X group was similar to the US1X group at 28 hours and showed a 1.2-fold significant decrease (P value <0.01) compared to the untreated group. These results indicate that the US protocols used in this study, which proved to be safe for healthy brain tissue, significantly inhibited the tumor growth in a very short time. Additionally, the two US protocols showed an essentially similar inhibition rate. It is possible however, that the 4-hours' time-interval was too short to observe any significant changes in tumor volume.

Tumor tissues histopathology evaluation of the control group is demonstrated in FIG. 3D, showing the tumor region (left image) and enlargement of the marked area in the tumor (right), 9 days after tumor implantation (control group). Scale bars equal to 2000 μm (left image) and 500 μm (blown-up image), respectively. The control tumor (denoted by “No US” is shown as a darker mass by an arrow (denoted as “Tumor”). In the demonstrated slide, the tumors consisted of compact cellular mass, composed of uniform dense population of cancerous cells without necrosis. The margins of the tumor tissue were clear and sharp, with no evidence of edema or penumbra in the adjacent tissue. The tumor tissue of the US treated groups presented a very clear difference in terms of structural changes as function of different tumor volumes. The percentage of the histologically characterized US-affected area (out of total tumor cross-section area), evaluated at the mid-coronal section of the tumor, was plotted as a function of the tumor volume, as demonstrated in FIG. 3E. In the Figure the percentage of the coronal area affected by US irradiation out of the total mid-coronal section tumor area (denoted by the label “Affected coronal area/total coronal area [%]” on ordinate axis) as a function of the tumor volume (denoted by the label “Tumor volume [mm³]” on the abscissa axis), for all US experimental groups which were histologically evaluated (4 groups: 4, 12, 28 hours US1X, and 28 hours US2X; n=6 per group). Coronal sections of representative rat brains which were analyzed, one from the extensive effect region (marked in darker hue, denoted as “Extensive”) and one from the superficial effect region (marked in lighter hue, denoted as “Superficial”). Scale bar on both images below the graph are 1000 μm. The areas are delineated with a thick solid line for total coronal area (denoted as “Total coronal area”), and by a thinner dashed line for affected coronal area (denoted as “Affected coronal area”). Two distinct groups could be observed. The first group includes tumor volumes higher than a cutoff value of 16 mm³, in which the US effect was superficial with less than 40% of the tumor being affected (as shown by the right-hand side of FIG. 3E). The second group includes tumor volumes smaller than 16 mm³, where the US effect is extensive, covering between 60-100% of the tumor area (as shown by the left-hand side of FIG. 3E).

It is important to note that even though the structural changes were minor for volumes higher than 16 mm³, an inhibition of tumor growth was observed. Importantly, this cutoff volume of 16 mm³ may probably be a value which is dependent on, and only relevant to, the US condition used in this study, i.e., the sonotrode parameters, the US frequency, intensity, and insonation time.

Charting the coronal tumor area normalized by the sonotrode transmitting surface area versus the percentage of coronal affected area is shown in FIG. 3F. The percentage of the affected coronal area by US irradiation from the total mid-coronal section tumoral area (denoted in the ordinate axis like in FIG. 3E) as a function of the ratio of total coronal tumor area to the US probe surface area (denoted as the abscissa axis label “Total coronal area/probe area”), for all US experimental groups which were histologically evaluated (4 groups: 4, 12, 28 hours US1X, and 28 hours US2X; n=6 per group), are presented. Thin dashed line represents the transition between extensive and superficial regions. The results indicate that the sonotrode area is an important factor; without being bound by a theory it is believed that the US radial expansion was limited under the experimental conditions and only the directly irradiated tumor tissue responded to the treatment.

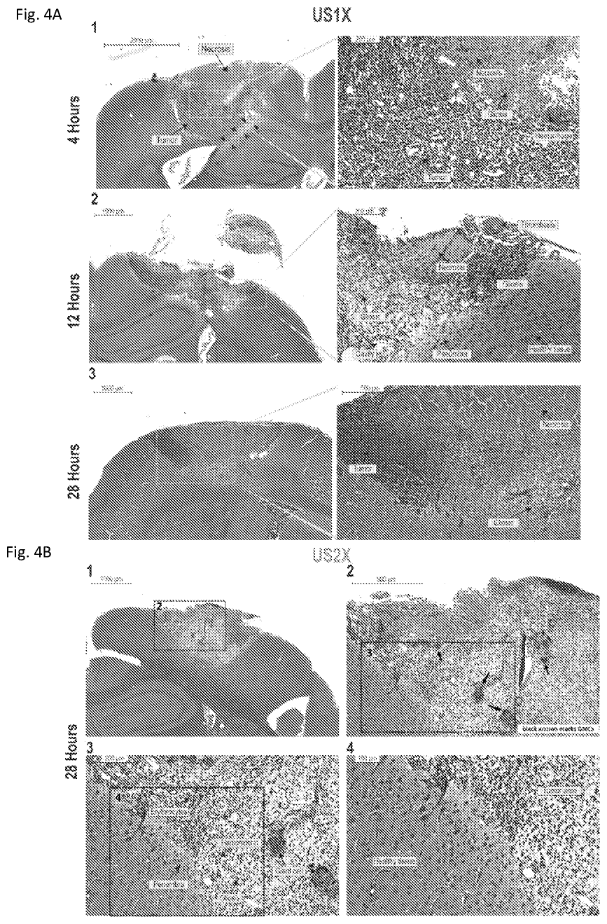

Representative histopathological images of smaller tumors are presented in FIG. 4. In the Figure, various features are observed, such as the tumor itself (denoted as “Tumor” and as “Tumor area” in FIG. 4B4), healthy tissue (denoted as “Healthy tissue”), necrosis area (denoted as “Necrosis”), gliosis area (denoted as “Gliosis”), hemorrhage sites (denoted as “Hemorrhage”), cavities (denoted as “Cavity”), insonation penumbra area (denoted as “Penumbra”), thrombosis events (denoted as “Thrombosis”), the presence of erythrocytes (denoted as “Erythrocytes”), of giant cells (denoted as “Giant cells”), and hemosiderin (denoted as “Hemosiderin). FIG. 4A shows evaluations of the structural changes resulting from treating the tumors with US1X protocol (denoted as “US1X”), at the indicated different time points post application, of which FIG. 4A1 shows different magnifications of coronal brain section from a rat bearing a 14 mm³ intracranial 9 L tumor, 4 hours after US irradiation was applied. Histological characteristics are marked within the images. Scale bar=2000 μm (left image) and 500 μm (right image); FIG. 4A2 shows different magnifications of a coronal brain section of rat bearing a 3 mm³ 9 L tumor, 12 hours after US irradiation was applied. Histological characteristics are marked within the images. Scale bar=1000 μm (left image) and 500 μm (right image); and FIG. 4A3 shows different magnifications of coronal brain section of rat bearing a 9 mm³ 9 L tumor, 28 hours after US irradiation was applied. Histological characteristics are marked within the images. Scale bar-1000 μm (left image) and 500 μm (right image). FIG. 4B shows evaluation of the structural changes resulting from treating the tumors with US2X protocol (denoted as “US1X”), 28 hours after the first application. FIG. 4B1-FIG. 4B4 show different magnifications of a coronal brain section of a rat bearing a 6 mm³ 9 L tumor after treatment with US2X (magnifications as marked in the images). Histological characteristics are marked within the images (MGCs is multinucleate giant cells). Scale bar=1000 μm (top left image), 500 μm (top right image), 200 μm (image A), 100 μm (image B). For the group that was treated with US1X (FIG. 4A), the tumor tissue was examined 4, 12 and 28 hours after US treatment. At 4 hours, a large necrotic area was found close to the brain's surface (FIG. 4A1). The necrotic features included an area of no cells, and in between ghost cells (cells without nucleus), necrotic cells, and erythrocytes. In addition, inflammatory cells were noted. Since the location of the necrosis is at the surface of the tumor, it indicates that it was caused by an external exposure and may therefore be attributed to the US radiation. In addition, at the margin of the tumor area, under the necrosis (its outlines are marked with black arrowheads), penumbra region can be seen with spongiosis (vacuolation). At 12 hours (FIG. 4A2), most of the tumor area was replaced with necrosis and glial reaction. An extensive gliosis can be found along with thrombosis, edema, hemorrhage, and mineralization (FIG. 5A1 and FIG. 5A2). In the FIG. 5A, evaluation of the structural changes resulting from treating the tumors with the US1X protocol (denoted as USIX″) is shown, at different magnifications of coronal brain section of rat bearing a 15 mm³ 9 L tumor, 12 hours after US irradiation was applied. Histological characteristics are marked within the images. Scale bar=1000 μm (left image) and 500 μm (right image). FIG. 5B shows evaluation of the structural changes resulting from treating the tumors with the US2X protocol (denoted as “US2XS”), at different magnifications of coronal brain section of rat bearing a 9 mm³ 9 L tumor after treatment with US2X. Histological characteristics are marked within the images. Scale bar=1000 μm (left image) and 200 μm (right image). Various features observed therein are same as in FIGS. 4A and 4b, with an additional feature of mineralization (denoted as “Mineralization”). In addition, cavities were formed as a result of the clearance of the necrotic tissue by the glial reaction. The tumor was surrounded by penumbra region, with adjacent healthy neurons. These results indicate that remarkably, 12 hours post US1X, the entire tumor was destroyed, while the healthy surrounding tissue was left unharmed. In tumors in which the volume was close to 16 mm³ (FIG. 5A), small areas of live tumor tissue were still left on the margins located at the sides of the tumor. Investigating the tumor tissues 28 hours after US treatment (FIG. 4A3), the same pattern was observed, in which most of the tumor area was replaced with necrosis and glial reaction. In the center of the tumor, necrotic area could be found, involving over 90% of the tumor area. In the margins of this area, infiltration of glial cells was noted, clearing the necrotic cells. On the margins located at the sides, small areas of live tumor tissue were also observed.

Remarkable results were achieved when the group that was treated with US2X was examined (FIG. 4B). The tumor tissue was examined 4 hours after the second application (namely, 28 hours after the first application). All of the tumor area was replaced with necrosis and extensive glial reaction (FIG. 4B1). In some brain's samples mineralization was also found (FIG. 5B1 and FIG. 5B2). The necrotic reaction is very small due to the infiltration of glial cells. No remnants of tumor cells were noted. An extensive gliosis was identified along with multinucleated giant cells (GMCs) engulfing the necrotic tumoral tissue (FIG. 4B2-FIG. 4B3). The extensive glial reaction and the presence of GMCs is indicative of the high rate of elimination of the necrotic tissue. In addition, erythrocytes and hemosiderin were noted in the necrotic area, indicative of hemorrhage. Moreover, cavities were formed, probably as a result of the necrotic tissue clearance by the glial reactive cells (FIG. 4B3). Surrounding the tumor tissue, there was a penumbra region and small area of gliosis, and externally to this, healthy neural tissue was seen (FIG. 4B3-FIG. 4B4). These results indicate that the entire tumor was eliminated by the US treatment, while the healthy tissue surrounding the tumor remained unaffected. According to this result the US treatment was highly effective and localized.

Overall, evaluation of the prominent features of the morphological reaction to US irradiation may be summarized as follows: 4 hours after US1X, the most evident is the necrotic reaction, indicating that at this time point, the morphological reaction is mainly acute. However, glial reaction is also present in the tumor area. Next, at 12 hours and 28 hours after US1X, the necrotic tissue is much reduced in size and there is a replacement by the glial reaction clearing the necrotic cells and leaving cavities in the affected area. When the US2X protocol was used, 28 hours after the first US irradiation and 4 hours after the second irradiation, the glial reaction was transforming into multinucleate giant cells (MGCs), and the elimination process of the necrotic tissue was accelerated. In general, the development of the morphological reaction between 4-28 hours was very fast, transforming from acute to subacute. This fast development of the reaction was correlated with the rapid inhibition of tumor growth seen 12 hours after US application.

Surprisingly, induction of glial reaction was observed only in the groups that were treated with US and not in the untreated groups. Furthermore, extensive gliosis was observed when US was applied twice. Without wishing to be bound by theory, these findings indicate that US or US-mediated effects on the tumor may induce an anti-tumor immune response. Necrosis and blood vessel disruption can trigger the immune system, consequently stimulating an inflammatory response. This may explain why the application of US2X was apparently more effective than US1X.

The US conditions used exhibited high selectivity since healthy tissue was not damaged. Regarding thermal effects, since calculation of TI in the safety study was at the safe range and led to a less than 1 °C increase in temperature, it is assumed that the main mechanisms which demonstrated a biological effect were non-thermal. It has now been therefore unexpectedly found that it is possible to affect transplanted GBM cancers in-vivo at diagnostically safe parameters by semi-invasive insonation.

### Example 4

Histological Assessment of Rat Brain with a Focal Cortical Lesion Treated with Ultrasound-Evaluation of Lesion Characteristics

Further to the above results, indicating a beneficial effect for insonation on brain tumors, the effect of ultrasound was also evaluated in a brain tissue injury model. Specifically, the histological objective of this study was to evaluate the effect of ultrasound treatment, on cortical lesions caused by skull drilling within the cerebrum of rat brains. The ultrasound treatment was applied shortly after the skull was drilled, as detailed below, and evaluation was performed on seven-day lesions.

Brain injury was induced using a published protocol (Buaron, N. et al. 2021 Advanced Functional Materials, Vol. 31, Issue 44; https://doi.org/10.1002/adfm.202100643). Briefly, rats were anesthetized, the head was shaved, and a midline scalp incision was made in order to expose the sagittal and coronal sutures. A hole of 3 mm was made in the skull, using an electric drill, centered 3 mm lateral to the sagittal suture and 5 mm posterior to the coronal suture. The skin was then closed with surgical staples. All surgical procedures were performed using standard sterile surgical technique.

After induction of brain injury, US was applied as follows. Animals (n=6) were placed in a custom-made head-stabilizing stand, and the US probe was inserted into a gel filled cylinder right on top of the burr hole. US at 20 kHz (Q125 Sonicator, Qsonica L.L.C, Newtown, CT, USA) with a probe tip diameter of 3.2 mm was used for application of US in a continuous mode, 10-60 minutes after the skull was drilled and while the animals were under full anesthesia. The applied US conditions were an intensity of 4.2 W/cm² for a period of 5 minutes. The skin was then closed with skin glue (3M Vetbond™ tissue adhesive). As a control, the same surgical protocol was followed, apart from the US application.

Brain tissues were then examined as detailed below, in a blinded manner by a pathologist (namely, without knowing which of the animals were treated by ultrasound or evaluated as a control group).

Histopathological characterization of the injury area was performed seven days after inducing the injury and application of US, by carefully removing the brains, fixing using 4% paraformaldehyde, and embedding in paraffin to obtain coronal sections of 10 μm. The levels of lymphocytes, gliosis, neovascularization and granulation in rat brain tissue (bearing brain injury) were evaluated as follows. For evaluation of the level of lymphocytes, gliosis and granulation, sections were than stained with hematoxylin/eosin (H&E). Granulation tissue grade was evaluated by a pathologist in a common semi-quantitative analysis. Quantitative evaluation of lymphocytes infiltration and gliosis was performed using morphometry analysis. Image Pro Plus Ver. 6.03 by Media Cybernetics (MediaCY) was used for the analysis. Spatial calibration and Area of Interest (AOI) were applied. Image segmentation (2 classes Threshold) and analysis was done by using the RGB histogram in order to depict and count the range of cells' types. The threshold depicted cells, (2 classes based on size and stain density), were included in the range statistics table, the results were transferred to the Data Collector and then to Excel. For the evaluation of neovascularization, sections were stained with CD34 staining. Quantitative evaluation was performed using morphometry analysis. Image analysis of the blood vessels was done with Image Pro Plus software by Media Cybernetics, Ver 6.3, (USA). AOI-Area of interest was chosen to depict and measure viable areas only within the AOI. The parameter SubArea was calculated from Area/AOI. Pictures were taken using microscope (Olympus BX60, serial NO. 7D04032) at magnification of ×1.25, and ×10, and microscope's Camera (Olympus DP73, serial NO. OH05504).

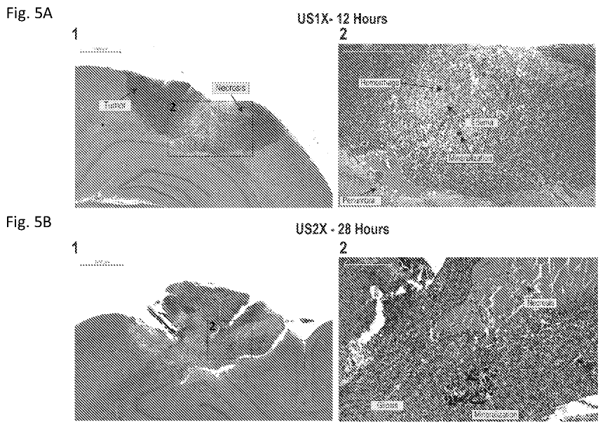

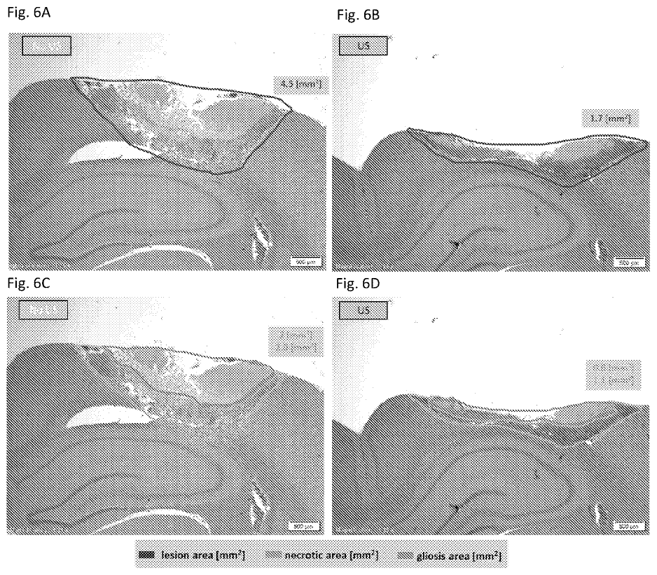

An exemplary histological evaluation of the lesion, in the absence of US application (control), is shown in FIG. 6. In the Figure, FIG. 6A shows intracranial lesion site without US (denoted as “No US”) (lesion's location and its calculated area (denoted as “4.5 [mm²]”) are marked); FIG. 6B intracranial lesion site after US application (denoted as “US”) (lesion's location and its calculated area (denoted as “1.7 [mm²]”) are marked). FIG. 6C-D demonstrate the different regions (necrosis and gliosis) found in the lesions, which are presented in FIGS. 6A and 6B, respectively (regions and their calculated area are marked); area was measured using ImageJ software. Scale bar-500 μm, magnification times: ×1.25. The areas are delineated with the solid lines, with the darkest delineating the lesion area (denoted as “lesion area [mm²]”), the lightest line the necrotic area which is present in the upper parts of FIGS. 6C and 6D (denoted as “necrotic area [mm²]”), the intermediate hue line the gliosis area which is present in the lower parts of lesions as seen in FIGS. 6C and 6D (denoted as “gliosis area [mm²]”). FIG. 6A and FIG. 6C. Exemplary histological evaluation of the lesion, in the presence of and US application at 4.2 W/cm² for 5 minutes is shown in FIG. 6B and in FIG. 6D. The lesion area (marked in red in FIG. 6A and in FIG. 6B can be characterized by two main regions: necrosis (marked in blue in FIG. 6C and in FIG. 6D) and gliosis (marked in green in FIG. 6C and in FIG. 6D. The gliosis region can be seen surrounding the necrotic region. When comparing the area of the lesions (i.e., of FIG. 6A and FIG. 6B), it can be seen that after US application the lesion area is much reduced (~60%). In addition, the necrotic area of the lesion when US was applied was smaller, and the gliosis region is the dominant region (FIG. 6C and FIG. 6D).

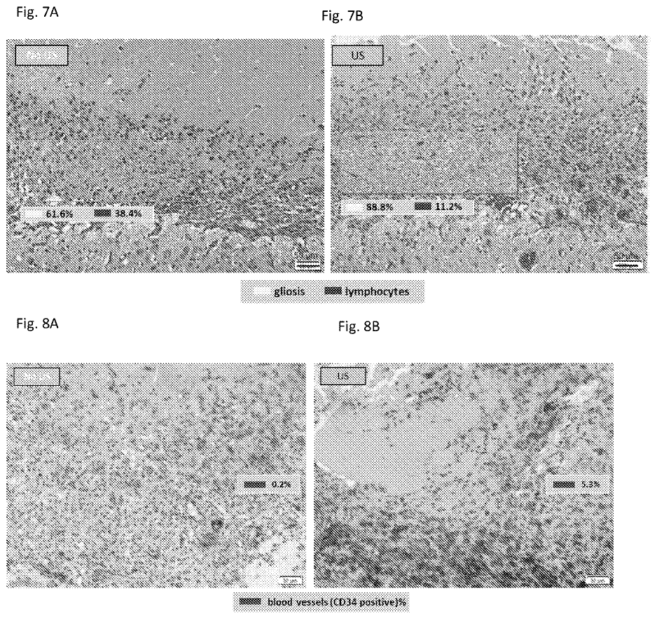

Next, brain tissues of rats were evaluated for the presence of inflammatory cells in response to the injury. Specifically, the levels of lymphocytes and gliosis were evaluated as detailed above. The lymphocytes infiltration and gliosis were compared quantitatively using morphometry analysis, as can be seen in FIG. 7A (obtained for a control sample) and in FIG. 7B (obtained upon US application). In the Figures, FIG. 7A shows the analysis of the composition of inflammatory cells in gliosis region without US (denoted as “No US”) (cells composition are marked with lighter color corresponding to gliosis, denoted as “gliosis”, and darker color corresponding to lymphocytes, denoted as “lymphocytes”); FIG. 7B shows the analysis of the composition of inflammatory cells in gliosis region after US application (denoted as “US) (cells composition are marked). Quantitative evaluation of lymphocytes infiltration and gliosis was performed using morphometry analysis. Scale bar=50 μm, magnification times: ×10. This analysis indicates an increase in the percentage of the gliosis cells and decrease in the percentage of the lymphocytes when US was applied. Without wishing to be bound by theory this observation means that there is less cellular inflammatory cells infiltration into the lesion when US was applied. Damaged neuronal tissue release cytokines and chemokines which triggers inflammatory response. The cellular inflammatory cells will infiltrate into the brain in response to a cascade of immune events controlled by the gliosis cells (e.g. microglia and astrocytes). Smaller levels of cellular inflammatory cells may indicate that US prevented the spreading of the damaged neuronal tissue, leading to smaller levels of infiltration of these cells (e.g. lymphocytes). This correlates with the smaller area of necrosis shown in FIG. 6 when US was applied. An additional explanation may be that US accelerated the healing process to a stage where the level of lymphocytes was declined in comparison to gliosis cells.

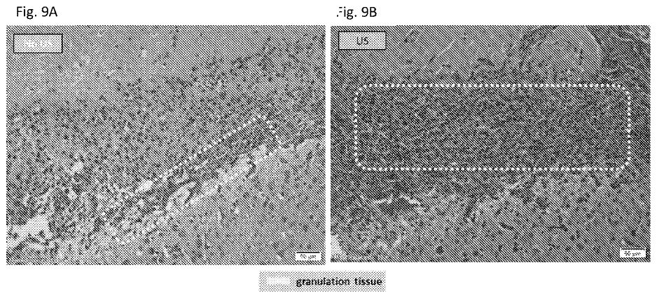

Brain tissues were further evaluated for neovascularization around the lesions, as detailed above. Interestingly, morphometry analysis showed enhancement in angiogenesis when US was applied (as shown in FIG. 8B) in comparison to control samples not subjected to US treatment (as shown in FIG. 8A). FIG. 8A shows the distribution of neovascularization in the gliosis region without US (denoted as “No US”) (% CD34 positive area is marked, denoted as “blood vessels (CD34positive) %”); FIG. 8B shows the distribution of neovascularization in the gliosis region after US application (denoted as “US”) (area is marked); Scale bar=50 μm, magnification times: ×10. Neovascularization is a key element in the healing process. The formation of new blood vessels facilitates the elimination of damaged cells and debris from the injured area and enables the delivery of oxygen and nutrients to the lesion site leading to acceleration of the healing process. One of the known indications for the acceleration of the healing process is the formation of a granulation tissue. US-treated and control injured brain tissues of rats were therefore further evaluated for granulation tissue formation, as detailed above. Evaluation of the granulation tissue shows that without US, small level of granulation tissue can be found (as shown in FIG. 9A). However, upon application of US, a dense granulation tissue, predominated by macrophages and multinucleated giant cells, which eliminates the injured tissue, can be found in the gliosis region (FIG. 9B). In the Figures, FIG. 9A shows the area of granulation tissue in the gliosis region without US (denoted as “No US”) (area is marked, denoted as “granulation tissue”); FIG. 9B shows the area of granulation tissue in the gliosis region after US application (denoted as “US”) (area is marked); Scale bar=50 μm, magnification times: ×10. Without wishing to be bound by theory this observation means that the healing process was accelerated when the lesion was treated with US.

The overall investigation suggests that application of US under the condition used can have a beneficial effect on the lesion healing process, prevent the spreading of necrosis and enable a faster repair of the tissue.

1. A method of treating a subject suffering from a primary cancer of a brain, said method comprising directly irradiating the cancerous lesion or non-resected tumor margins in a post-operative cavity with ultrasound having a frequency of between 20 and 200 kHz, at an ultrasound intensity of between 1 and 50 W/cm², and wherein said ultrasound intensity is characterized by a thermal index (TI) value of between 0.1 than 5 and a low-frequency mechanical index (LFMI) value between 0.15 than 3.

2. The method according to claim 1, wherein said directly irradiating is performed by placing the ultrasound transducer onto said cancerous lesion or into said post-operative cavity.

3. The method according to claim 1, wherein said frequency is between 20 kHz and 35 kHz.

4. The method according to claim 1, wherein said intensity is between about 1 and 5.5 W/cm² and the frequency is between 20 and 22 kHz.

5. The method according to claim 1, wherein ultrasound is applied at least once for a time interval of between 5 seconds and 30 minutes.

6. The method according to claim 1, wherein said primary brain cancer is selected from disease or disorders classified in a subclass 02A00, 02A01, 02A02, or 02A0Z, of the 11th revision of World Health Organization International Classification of Diseases.

7. The method according to claim 1, wherein said frequency and intensity are selected such that the thermal index value is selected from the group consisting of between 0.1 and 5.0, between 0.1 and 4.5, between 0.1 and 4.0, between 0.1 and 3.5, between 0.1 and 3.0, between 0.1 and 2.5, between 0.1 and 2.0, between 0.1 and 1.5, and between 0.1 and 1.0, and said low-frequency mechanical index value is selected from the group consisting of between 0.15 and 3.0, between 0.15 and 2.8, between 0.15 and 2.6, between 0.15 and 2.5, between 0.15 and 2.4, between 0.15 and 2.2, between 0.15 and 2.0, and between 0.15 and 1.9.

8. A method of treating a subject suffering from a brain injury, said method comprising directly irradiating an area of said brain injury with ultrasound having a frequency of between 20 and 200 kHz, at an ultrasound intensity of between 0.3 and 50 W/cm², wherein said ultrasound intensity is characterized by a thermal index (TI) value lower than 5.0 and a low-frequency mechanical index (MI) value lower than 3.

9. The method according to claim 8, wherein said thermal index value is selected from the group consisting of lower than 5.0, lower than 4.5, lower than 4.0, lower than 3.5, lower than 3.0, lower than 2.5, lower than 2.0, lower than 1.5 and lower than 1.0, and said low-frequency mechanical index value is selected from the group consisting of lower than 3.0, lower than 2.8, lower than 2.6, lower than 2.5, lower than 2.4, lower than 2.2, lower than 2.0, and lower than 1.9.

10. The method according to claim 8, wherein said brain injury results from traumatic brain injury.

11. The method according to claim 8, wherein said directly irradiating is performed between 10 minutes and 48 hours post injury.

12. A method of treating a subject suffering from a cancer in an organ or a tissue, said method comprising directly irradiating said organ or tissue with ultrasound having a frequency of between 20 and 200 kHz, at an ultrasound intensity of between 1 and 50 W/cm² and wherein said ultrasound intensity is characterized by a thermal index (TI) value of higher than 0.1 and lower than 5, and a low-frequency mechanical index (MI) value of higher than 0.15 and lower than 3.

13. The method according to claim 12, wherein said directly irradiating is performed by placing the ultrasound transducer in the proximity of said tissue or organ.

14. The method according to claim 12, wherein said directly irradiating is performed by placing the ultrasound transducer within a cavity formed by, or during a surgery in the vicinity of said organ or tissue.

15. The method according to claim 12, wherein said frequency is between 20 kHz and 35 kHz.

16. The method according to claim 12, wherein said intensity is between about 1 and 5.5 W/cm² and the frequency is between 20 and 22 kHz.

17. The method according to claim 12, wherein said intensity is between 4 and 5 W/cm² and the frequency is between 20 and 22 kHz.

18. The method according to claim 12, wherein ultrasound is applied at least once for a time interval of between 5 seconds and 30 minutes.

19. The method according to claim 12, wherein ultrasound is applied multiple times, at a regimen frequency of between once or twice daily to once weekly, during a period of between one to four weeks.

20. The method according to claim 12, wherein ultrasound is applied at an angle of between about −60° and about 60° relative to a perpendicular axis to said organ or tissue surface.

21. The method according to claim 12, wherein said method is an adjuvant therapy to surgery of said organ or tissue.

22. The method according to claim 21, for eliminating non-resected margins of said cancer in said organ or said tissue after said surgery, or for eliminating metastatic cells.

23. The method according to claim 12, wherein said cancer is a brain metastasis of a cancer of different origin.

24. The method according to claim 12, wherein said cancer is selected from disease or disorders classified in any one of subclasses 02A20 to 02F9Z, of the 11th revision of World Health Organization International Classification of Diseases, preferably selected from the group consisting of a sarcoma, a carcinoma, a lymphoma, a cancer of the breast, of the ovary, of the lung, of the prostate, of the colon, of the skin, and of the bladder.

25. The method according to claim 12, wherein said thermal index value is selected from the group consisting of between 0.1 and 5.0, between 0.1 and 4.5, between 0.1 and 4.0, between 0.1 and 3.5, between 0.1 and 3.0, between 0.1 and 2.5, between 0.1 and 2.0, between 0.1 and 1.5 and between 0.1 and 1.0, and said low-frequency mechanical index value is selected from the group consisting of between 0.15 and 3.0, between 0.15 and 2.8, between 0.15 and 2.6, between 0.15 and 2.5, between 0.15 and 2.4, between 0.15 and 2.2, between 0.15 and 2.0, and between 0.15 and 1.9.
